(19) United States
(12) Patent Application Publication
(10) Pub. No.: US 2025/0191002 A1
(43) Pub. Date: Jun. 12, 2025

(54) SYSTEM FOR DETERMINING DEGREE OF INTEREST, SERVER DEVICE, AND METHOD FOR DETERMINING DEGREE OF INTEREST

(71) Applicant: SEKISUI HOUSE, LTD., Osaka-shi, Osaka (JP)

(72) Inventors: Minami YAMAZAKI, Osaka (JP); Satoshi OHTA, Osaka (JP); Yasuyo IWAMAE, Osaka (JP); Ikuto UEYAMA, Osaka (JP); Chiho HIRAOKA, Osaka (JP); Masayuki KONDO, Osaka (JP)

(73) Assignee: SEKISUI HOUSE, LTD., Osaka-shi, Osaka (JP)

(21) Appl. No.: 18/274,107

(22) PCT Filed: Dec. 27, 2021
(86) PCT No.: PCT/JP2021/048618

§ 371 Date: Nov. 18, 2024

(30) Foreign Application Priority Data
Jan. 29, 2021 (JP) 2021-013152

(51) Int. Cl. G06Q 30/0201; A61B 5/16; G06Q 50/16

(52) U.S. Cl. CPC G06Q 30/0201; A61B 5/167; G06Q 50/16

(57) ABSTRACT

A server device (10) includes a reception part (22) that receives physiological information, location information, and action information of a guest, and an interest degree analysis section (33) that determines, on the basis of the physiological information, the location information, and the action information received by the reception part (22), an interest degree of the guest with respect to a building, and outputs interest degree information indicative of the interest degree.

### TECHNICAL FIELD

The present invention relates to interest degree determination system, server device, program, and an interest degree determination program.

### BACKGROUND ART

In a housing exhibition site including a plurality of model houses, there is a service that a salesperson (a host) of a housing manufacturer accompanies and guides a visitor (a guest) to the model houses in turn. The host analyses an interest degree of the guest with respect to a building on the basis of a preliminary questionnaire, a conversation with the guest during the guidance, or the like, and utilizes collected information to conduct the guidance service and subsequent sales activity with respect to the guest.

The following Patent Literature 1 discloses a person trend recording device that records customer trends at a clothing store in a shopping mall. The person trend recording device includes an image acquisition part that acquires moving image data obtained by taking images of a target area, a person detection part that detects and tracks a person who acts in the target area by analyzing the moving image data, an event detection part that detects an event occurred to the person in a period in which the person is tracked by analyzing the moving image data, a physio-psychological index detection part that detects a chronological change in a physio-psychological index of the person during the tracking period, a satisfaction degree estimation part that estimates a satisfaction degree of the person on the basis of the chronological change in the physio-psychological index, and a trend record generation part that generates, as trend record data on the person, data in which at least one or more events occurred to the person, the chronological change in the physio-psychological index of the person, and information concerning the satisfaction degree estimated by the satisfaction degree estimation part are associated one another with respect to time.

In the guidance service provided in the housing exhibition site that is mentioned in the Background Art, the guest is likely not to state a true intention in the preliminary questionnaire. Additionally, amount and quality of information that each host is capable of collecting from the guest significantly vary depending on a conversation skill of the host at taking out necessary information from the guest during the conversation, an analytical skill of the host at grasping the true intention of the guest on the basis of an utterance of the guest, and the like.

### CITATION LIST

### Patent Literature

Patent Literature 1: Japanese Patent No. 6724827

### SUMMARY OF INVENTION

The present invention has been made in view of the circumstances described above, and an object thereof is to obtain interest degree determination system, server device, program, and interest degree determination method, which enable collection of accurate interest degree information indicative of an interest degree of a guest with respect to a building.

An interest degree determination system according to an aspect of the present invention is an interest degree determination system for determining, in a service allowing a guest to observe an inside of at least one building, an interest degree of the guest with respect to the building, which includes a server device, a first sensor that detects physiological information concerning the guest and sends the physiological information to the server device, a second sensor that detects a location of the guest in the building and sends location information indicative of the location to the server device, and a third sensor that detects a predefined action of the guest in the building and sends action information indicative of the action to the server device. The server device includes a first reception part that receives the physiological information, the location information, and the action information, and an analysis part that determines, on the basis of the physiological information, the location information, and the action information received by the first reception part, an interest degree of the guest with respect to the building, and outputs interest degree information indicative of the interest degree.

### BRIEF DESCRIPTION OF DRAWINGS

FIG. 1 is a diagram showing a simplified general configuration of an interest degree determination system according to an embodiment of the present invention.

FIG. 2 is a diagram showing a simplified configuration of a server device.

FIG. 3 is a diagram showing a simplified configuration of a tablet.

FIG. 4 is a flowchart showing a process of setting a guidance order of a plurality of model houses.

FIG. 5 is a graph that schematically represents a part of received physiological information.

FIG. 6 is a flowchart showing a calculation process of basic point that constitutes a part of interest degree information concerning a guest.

FIG. 7 is a graph schematically representing received physiological information.

FIG. 8 is a table showing exemplary interest degree information concerning a guest to a model house.

FIG. 9 is a flowchart showing a calculation process of action point that constitutes a part of the interest degree information concerning the guest.

FIG. 10 is a flowchart showing a calculation process of individual point that constitutes a part of the interest degree information concerning the guest.

FIG. 11 is a flowchart showing a rest setting process by a rest determination section.

FIG. 12 is a flowchart showing an output process of result information indicative of a result of an interest degree determination concerning the guest.

FIG. 13 is a table showing exemplary related data stored in a storage part.

### DESCRIPTION OF EMBODIMENTS

Hereinafter, an embodiment of the present invention will be described with reference to the accompanied drawings. Elements allotted with the same sign in different drawings are construed to be the same element or corresponding elements.

### System Configuration

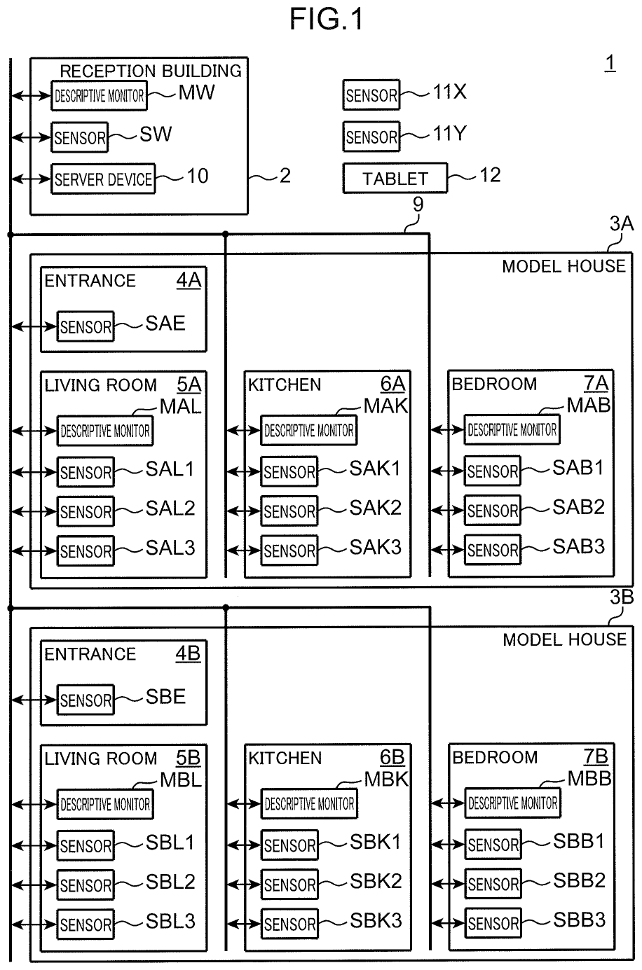

FIG. 1 is a diagram showing a simplified general configuration of an interest degree determination system 1 according to an embodiment of the present invention. The interest degree determination system 1 is applied to a housing exhibition site provided with a plurality of model houses 3. In the housing exhibition site, there is offered a guidance service, by a salesperson (a host) of a housing manufacturer or the like, of accompanying and guiding a visitor (a guest) to the model houses 3 in turn. Alternatively, the service may be offered in a style that the guest observes model houses 3 unassisted without being guided by a host.

In the example shown in FIG. 1, the housing exhibition site is provided with a reception building 2 where an arrived guest is received, and model houses 3A, 3B to be observed by the guest in turn. FIG. 1 shows only the two model houses 3A, 3B for the sake of conciseness of the description. However, the housing exhibition site may be provided with three or more model houses 3 having concepts similar to or different from one another.

A sensor 11 is lent to the guest at the reception building 2. The guest wears the borrowed sensor 11. When a group of guests such as a family arrives, each guest wears a sensor 11. For example, when a group of two guests arrives, one guest wears a sensor 11X, whereas the other guest wears a sensor 11Y.

The sensor 11 is, for example, a smartwatch wearable on a wrist. The sensor 11 is mounted with a physiological information sensor, a location sensor, a display part, and a wireless communication part. The physiological information sensor includes a heart rate monitor, a thermometer, a sweat meter, and the like, and detects physiological information (a heart rate, a body temperature, a sweat rate, and the like) of the guest wearing the sensor 11, and outputs the physiological information. The location sensor includes a GPS receiver, an RFID tag, and the like, and detects a location of the guest wearing the sensor 11 outdoors and indoors at the housing exhibition site to output location information indicative of the location. The display part includes a display including liquid crystal, organic EL, or the like, and displays a map of an inside of the housing exhibition site, an outline of each model house 3, and physiological information and the like of the guest wearing the sensor 11. The wireless communication part includes a communication module adapted for a desired wireless communication way, e.g., Wi-Fi and Bluetooth (registered trademark), and sends the physiological information output from the physiological information sensor and the location information output from the location sensor to a server device 10 together with discrimination information concerning the sensor 11 itself.

The host carries a portable terminal (operation terminal) such as a tablet 12 or a laptop personal computer. The host accompanies and guides the guest inside the housing exhibition site carrying the tablet 12. Details on a configuration of the tablet 12 will be described later.

The reception building 2 is provided with a descriptive monitor MW, a sensor SW, and the server device 10. The descriptive monitor MW is a monitor including liquid crystal, organic EL, or the like, and plays a recorded video that introduces an outline of each of the model houses 3 to thereby present a description concerning each model house 3 to the guest. The sensor SW includes an RFID reader or the like, and detects a sensor 11 existing in the reception building 2 via a communication with an RFID tag to output location information indicative of a current location of the sensor 11 in the reception building 2. Details on the configuration of the server device 10 will be described later. The server device 10 may be provided outside the reception building 2.

The model house 3A includes an entrance 4A and a plurality of observable areas including a living room 5A, a kitchen 6A, and a bedroom 7A. FIG. 1 shows an exemplary model house 3A in which only three observable areas are defined for the sake of conciseness of description. However, in the model house 3A, another observable area, e.g., a dining room or a children's room may be defined. The same applies to the other model house 3.

The entrance 4A is provided with a sensor SAE. The sensor SAE includes an RFID reader and the like, and detects a sensor 11 existing in the entrance 4A via a communication with an RFID tag to output location information indicative of a current location of the sensor 11 in the entrance 4A.

The living room 5A is provided with a descriptive monitor MAL and sensors SAL1 to SAL3. The descriptive monitor MAL includes a monitor having liquid crystal, organic EL, or the like, and plays a recorded video that introduces an outline of the living room 5A to thereby present a description concerning the living room 5A to the guest. In the recorded video, descriptions concerning the living room 5A are classified into a plurality of interest degree items (in the example described hereinafter, “Life Proposal”, Interior”, and “Furniture and Equipment”). Life Proposal is an item concerning a way of life, a lifestyle, or the like of the room. Interior is an item concerning a total atmosphere, a total coordination, or the like of the room. Furniture and Equipment is an item concerning a name of a manufacturer, a name of a model, or the like of a furniture, an electric appliance, or the like provided in the room.

The sensor SAL1 includes an RFID reader or the like, and detects a sensor 11 existing in the living room 5A via a communication with an RFID tag to output location information indicative of a current location of the sensor 11 in the living room 5A. Hereinafter, a sensor that detects a location of the sensor 11 is referred to as “a location sensor”.

The sensors SAL2, SAL3 are sensors that detect a preliminarily defined action (hereinafter, referred to as “predefined action”) of the guest in the living room 5A. For example, the sensor SAL2 includes a weight sensor that detects a predefined action of sitting on a sofa. The sensor SAL3 includes an electric current sensor that detects a predefined action of turning on an electric appliance. There may be provided another sensor that detects another predefined action of the guest. The same applies to the other observable areas. Hereinafter, a sensor that detects a predefined action of the guest is referred to as “an action sensor”.

Similarly to the living room 5A, the kitchen 6A is provided with a descriptive monitor MAK, a location sensor SAK1, and action sensors SAK2, SAK3. Further, the bedroom 7A is provided with a descriptive monitor MAB, a location sensor SAB1, and action sensors SAB2, SAB3.

Similarly to the model house 3A, in the model house 3B, an entrance 4B, a living room 5B, a kitchen 6B, and a bedroom 7B are defined. The entrance 4B is provided with a location sensor SBE. The living room 5B is provided with a descriptive monitor MBL, a location sensor SBL1, and action sensors SBL2, SBL3. The kitchen 6B is provided with a descriptive monitor MBK, a location sensor SBK1, and action sensors SBK2, SBK3. The bedroom 7B is provided with a descriptive monitor MBB, a location sensor SBB1, and action sensors SBB2, SBB3.

The descriptive monitors MW, MAL, MAK, MAB, MBL, MBK, MBB, the location sensors SW, SAE, SAL1, SAK1, SAB1, SBE, SBL1, SBK1, SBB1, and the action sensors SAL2, SAL3, SAK2, SAK3, SAB2, SAB3, SBL2, SBL3, SBK2, SBK3, SBB2, SBB3 are connected to the server device 10 via a desired wired or wireless communication network 9.

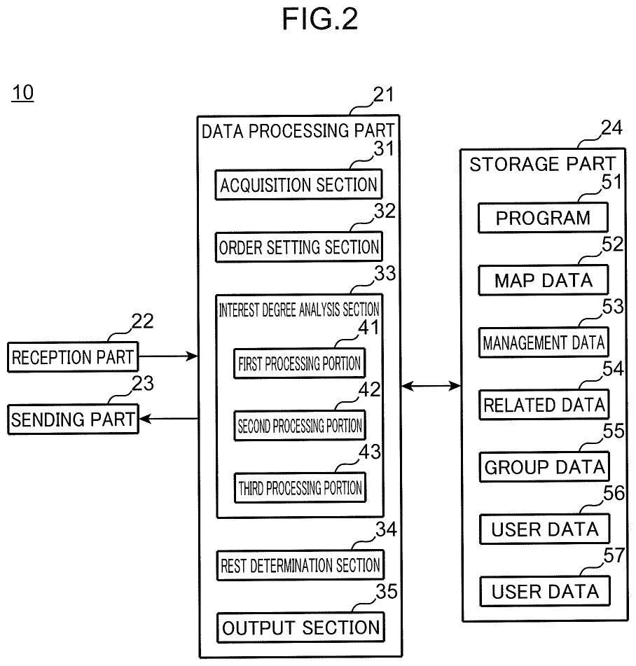

FIG. 2 is a diagram showing a simplified configuration of the server device 10. The server device 10 includes a data processing part 21, a reception part 22, a sending part 23, and a storage part 24. The data processing part 21 includes a CPU or the like. The reception part 22 and the sending part 23 include a communication module that allows the server device 10 to mutually communicate with the sensor 11, the tablet 12, the descriptive monitors MW, MAL, MAK, MAB, MBL, MBK, MBB, the location sensors SW, SAE, SAL1, SAK1, SAB1, SBE, SBL1, SBK1, SBB1, and the action sensors SAL2, SAL3, SAK2, SAK3, SAB2, SAB3, SBL2, SBL3, SBK2, SBK3, SBB2, SBB3, which are shown in FIG. 1. The storage part 24 includes HDD, SSD, Flash Memory, or the like. The storage part 24 stores a program 51, map data 52, management data 53, related data 54, group data 55, and user data 56, 57. The data processing part 21 includes an acquisition section 31, an order setting section 32, an interest degree analysis section 33, a rest determination section 34, and an output section 35 as functions performed by the CPU through an implementation of the program 51. The interest degree analysis section 33 has a first processing portion 41, a second processing portion 42, and a third processing portion 43. Details on the process executed by each part, section, and portion will be described later.

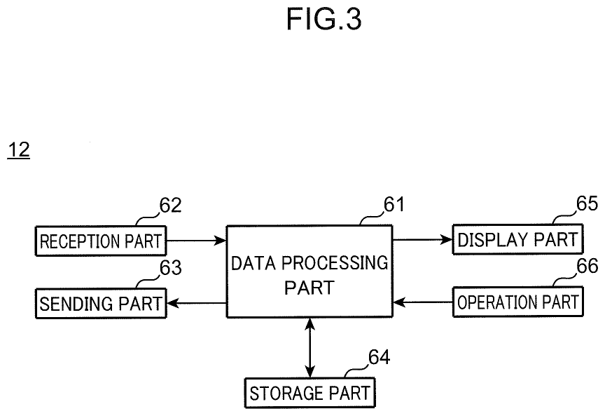

FIG. 3 is a diagram showing a simplified configuration of the tablet 12. The tablet 12 includes a data processing part 61, a reception part 62, a sending part 63, a storage part 64, a display part 65, and an operation part 66. The data processing part 61 includes CPU and the like. The reception part 62 and the sending part 63 include a communication module that allows the tablet 12 to mutually communicate with the server device 10. The storage part 64 includes Flash Memory and the like. The display part 65 and the operation part 66 include a touchscreen-type liquid crystal or organic EL display and the like.

### Guidance Order Setting Process

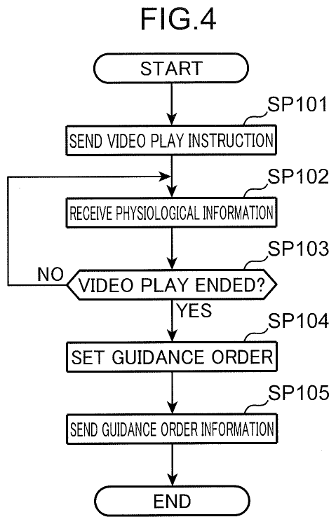

FIG. 4 is a flowchart showing a process of setting a guidance order of the model houses 3.

In the reception building 2, the host leads the guest wearing the sensor 11 to the descriptive monitor MW. The sensor SW detects the sensor 11, and sends location information indicative of a current location of the sensor 11 in the reception building 2 to the server device 10.

The storage part 24 of the server device 10 stores map data 52. The map data 52 has various location information including: (1) a location of the reception building 2 in the housing exhibition site; (2) a location of each model house 3 in the housing exhibition site; (3) locations of respective descriptive monitors MW, MAL, MAK, MAB, MBL, MBK, MBB; and (4) a location of furniture, an electric appliance, or the like that receives a predefined action in each observable area of each model house 3.

Further, the storage part 24 stores management data 53. The management data 53 has various management information including: (1) an association relationship between discrimination information concerning each sensor 11 and each guest; (2) an association relationship between discrimination information concerning a sensor 11 of each guest and discrimination information concerning a tablet 12 carried by the host who is in charge of the guidance of the guest; (3) discrimination information concerning a plurality of sensors 11 forming a group; and (4) progress information indicative of a progress of the guidance service for each guest.

The reception part 22 of the server device 10 receives the location information concerning the sensor 11 sent from the sensor SW. The acquisition section 31 acquires the location information concerning the sensor 11 from the reception part 22. The order setting section 32 determines with reference to the map data 52 and the management data 53 whether the presentation of the description to the guest staying before the descriptive monitor MW (i.e., the guest wearing the sensor 11) in the reception building 2 is completed. In a case where the presentation of the description is not completed, the order setting section 32 sends a video play instruction to the descriptive monitor MW in Step SP101. Specifically, the output section 35 outputs the video play instruction, and the sending part 23 sends the video play instruction that is input from the output section 35 to the descriptive monitor MW. The descriptive monitor MW receives the video play instruction, and plays a recorded video showing respective outlines of the model houses 3 provided in the housing exhibition site in turn. Accordingly, the recorded video is presented to the guest staying before the descriptive monitor MW.

While the descriptive monitor MW plays the recorded video, the sensor 11 detects physiological information concerning the guest, and continuously sends the detected physiological information to the server device 10 together with discrimination information concerning the sensor 11. In Step SP102, the reception part 22 of the server device 10 receives the physiological information sent from the sensor 11. The acquisition section 31 acquires the physiological information from the reception part 22. Since a play time of the recorded video by the descriptive monitor MW is known, the server device 10 repeatedly executes the processing of Step SP102 until the play of the recorded video ends.

When the play of the recorded video by the descriptive monitor MW ends (Step SP103: YES), next, in Step SP104, the order setting section 32 sets a guidance order of the model houses 3 to the guest on the basis of the physiological information concerning the guest received from the sensor 11.

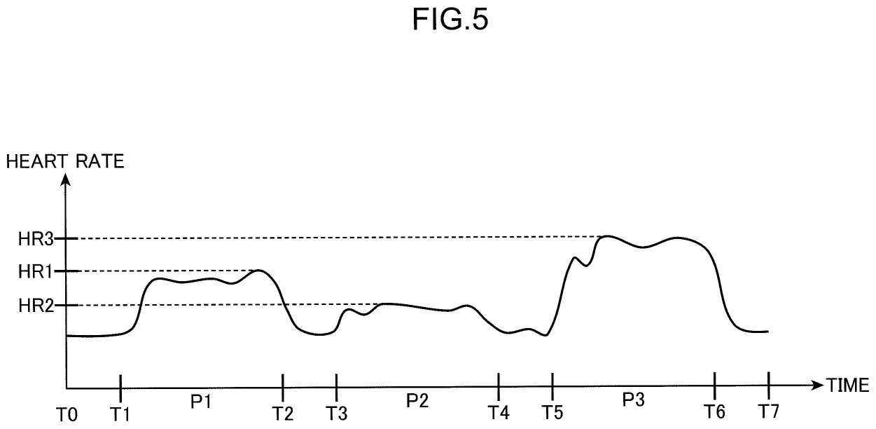

FIG. 5 is a graph that schematically represents a part of the received physiological information. The horizontal axis represents time, whereas the vertical axis represents a heart rate as exemplary physiological information. The descriptive monitor MW starts playing the recorded video at a time TO, gives an outline of a first model house 3 in a period P1 between a time T1 and a time T2, gives an outline of a second model house 3 in a period P2 between a time T3 and a time T4, and gives an outline of a third model house 3 in a period P3 between a time T5 and a time T6. Periods between the time T0 and the time T1, the time T2 and the time T3, the time T4 and the time T5, and the time T6 and the Time T7 constitute intervals.

The order setting section 32 finds heart rate maximum values HR1 to HR3 in the respective periods P1 to P3, and sorts the model houses 3 in a descending order of the maximum values HR1 to HR3 of the corresponding periods P1 to P3 to thereby set the guidance order of the model houses 3. In the example shown in FIG. 5, the order setting section 32 sets the guidance order as: the third model house 3→the first model house 3→the second model house 3. The physiological information used by the order setting section 32 to set the guidance order may include a body temperature, a sweat rate, and the like besides the heart rate. In the case where guests constitute a group, the order setting section 32 may set a guidance order on the basis of physiological information concerning a guest who is in the most leading position or most eager to acquire a house among the guests. The information is acquired through the preliminary questionnaire addressed to the group in the reception building 2, and input into the tablet 12 by the host, and sent to the server device 10, and then stored in the storage part 24 as the group data 55.

Next, in Step SP105, the order setting section 32 sends guidance order information indicative of the guidance order set in Step SP104 to the tablet 12 carried by the host who is in charge of the guidance of the guest. Specifically, the output section 35 outputs the guidance order information, and the sending part 23 sends the guidance order information input from the output section 35 to the tablet 12. The data processing part 61 of the tablet 12 displays the guidance order information received by the reception part 62 on the display part 65. The host guides the guest to the model houses 3 in the guidance order displayed on the display part 65 of the tablet 12.

### Generation Process of Interest Degree Information

Hereinafter, a generation process of interest degree information concerning the guest will be described by referring to an exemplary case where the host guides the guest to the model houses 3A. The interest degree information concerning the guest is stored in the storage part 24 as the user data 56.

When the guest enters an inside of the entrance 4A of the model house 3A under the guidance of the host, the location sensor SAE detects the sensor 11, and sends location information indicative of a current location of the sensor 11 inside the entrance 4A to the server device 10. Accordingly, the server device 10 starts the generation process of interest degree information concerning the guest with respect to the model house 3A.

### Calculation Process of Basic Point

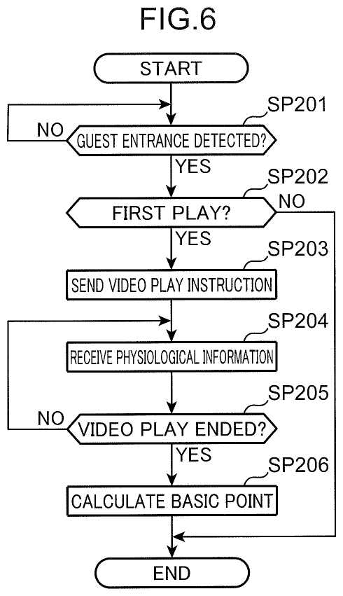

FIG. 6 is a flowchart showing a calculation process of basic point that constitutes a part of the interest degree information concerning the guest.

The host guides the guest from the entrance 4A to the living room 5A, and guides the guest to the descriptive monitor MAL in the living room 5A. The location sensor SAL1 detects the sensor 11, and sends location information indicative of a current location of the sensor 11 inside the living room 5A to the server device 10.

The reception part 22 of the server device 10 receives the location information concerning the sensor 11 from the location sensor SAL1. The acquisition section 31 acquires the location information concerning the sensor 11 from the reception part 22.

When detecting that the guest enters the living room 5A on the basis of the location information concerning the sensor 11 (Step SP201: YES), next, in Step SP202, the interest degree analysis section 33 refers to the map data 52 and the management data 53 to thereby determine whether the presentation of the description to the guest staying before the descriptive monitor MAL in the living room 5A is completed. Specifically, the interest degree analysis section 33 determines whether the play of the recorded video concerning the living room 5A constitutes the first play for the guest.

In a case where this constitutes the first play (Step SP202: YES), in Step SP203, the interest degree analysis section 33 sends a video play instruction to the descriptive monitor MAL. Specifically, the output section 35 outputs the video play instruction, and the sending part 23 sends the video play instruction input from the output section 35 to the descriptive monitor MAL. The descriptive monitor MAL receives the video play instruction, and plays a recorded video that gives an outline of the living room 5A of the model house 3A. Accordingly, the recorded video is presented to the guest staying before the descriptive monitor MAL. As described above, in the recorded video, descriptions concerning the living room 5A are classified into the interest degree items (i.e., “Life Proposal”, Interior”, and “Furniture and Equipment”).

While the descriptive monitor MAL plays the recorded video, the sensor 11 detects physiological information concerning the guest, and continuously sends the detected physiological information to the server device 10 together with the discrimination information concerning the sensor 11. In Step SP204, the reception part 22 of the server device 10 receives the physiological information from the sensor 11. The acquisition section 31 acquires the physiological information from the reception part 22. Since a play time of the recorded video by the descriptive monitor MAL is known, the server device 10 repeatedly executes the processing of Step SP204 until the play of the recorded video ends.

When the play of the recorded video by the descriptive monitor MAL ends (Step SP205: YES), in Step SP206, the first processing portion 41 of the interest degree analysis section 33 calculates a basic point of the guest to the living room 5A on the basis of the physiological information concerning the guest from the sensor 11. On the basis of the physiological information detected by the sensor 11 during the period in which the descriptive monitor MAL plays the recorded video, the first processing portion 41 allots the interest degrees of the guest to the living room 5A to each of the interest degree items (i.e., “Life Proposal”, Interior”, and “Furniture and Equipment”) and represents them in a numerical form to thereby obtain a basic point.

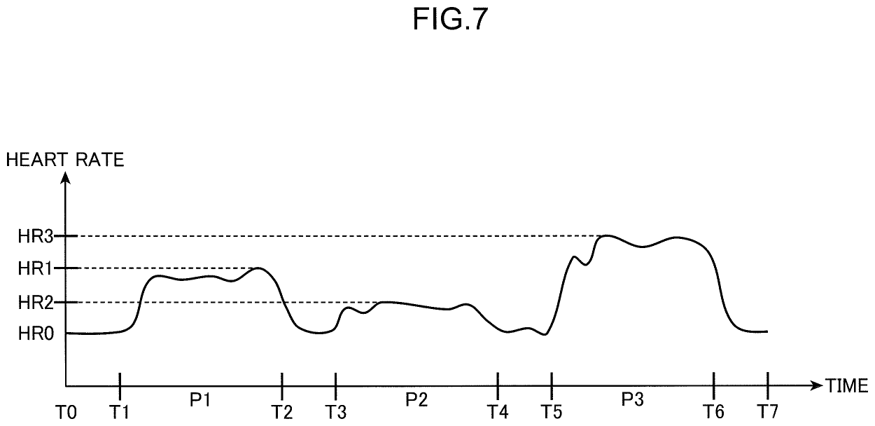

FIG. 7 is a graph schematically representing the received physiological information. The horizontal axis represents time, whereas the vertical axis represents a heart rate as exemplary physiological information. The descriptive monitor MAL starts playing the recorded video at a time TO, provides a description about the interest degree item “Life Proposal” in a period P1 between a time T1 and a time T2, provides a description about the interest degree item “Interior” in a period P2 between a time T3 and a time T4, and provides a description about the interest degree item “Furniture and Equipment” in a period P3 between a time T5 and a time T6. Periods between the time T0 and the time T1, the time T2 and the time T3, the time T4 and the time T5, and the time T6 and the Time T7 constitute intervals.

The first processing portion 41 calculates a heart rate average value HR0 in the intervals. Further, the first processing portion 41 finds heart rate maximum values HR1 to HR3 in the respective periods P1 to P3. Concerning the living room 5A, the first processing portion 41 allots the interest degree items basic points commensurate with the respective differences between the average value HR0 and the maximum values HR1 to HR3. The first processing portion 41 allots more basic points as the difference is larger, and assigns no basic point when the difference is equal to or smaller than a certain value. Further, the first processing portion 41 allots a basic point on the basis of another set of physiological information, e.g., a body temperature or a sweat rate besides the heart rate, in the same manner as the heart rate. The first processing portion 41 calculates a basic point for each interest degree item concerning the living room 5A by summing all the basic points, allotted on the basis of a plurality of sets of physiological information concerning the living room 5A, of each interest degree item.

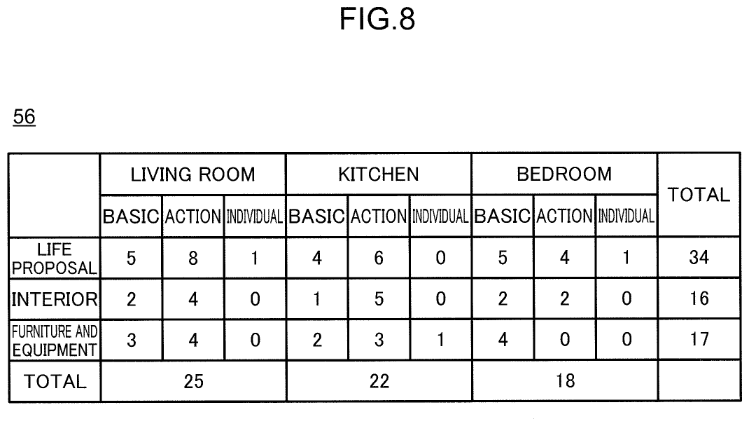

FIG. 8 is a table showing exemplary interest degree information (the user data 56) concerning the guest to the model house 3A. The basic points concerning the living room 5A are: five points for “Life Proposal”; two points for “Interior”; and three points for “Furniture and Equipment”, respectively.

In a case where the play of the recorded video concerning the living room 5A does not constitute the first play for the guest (Step SP202: NO), the first processing portion 41 does not execute the calculation process of basic point.

### Calculation Process of Action Point

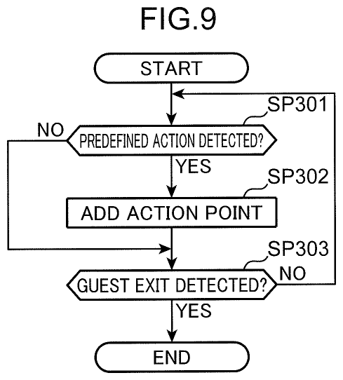

FIG. 9 is a flowchart showing a calculation process of action point that constitutes a part of the interest degree information concerning the guest.

While the guest stays in the living room 5A, the location sensor SAL1 continuously sends location information indicative of a current location of the sensor 11 in the living room 5A to the server device 10. When the guest takes a predefined action in the living room 5A, the action sensors SAL2, SAL3 detect the predefined action, and send action information indicative of the detection of the predefined action to the server device 10.

The reception part 22 of the server device 10 receives the action information sent from the action sensors SAL2, SAL3. The acquisition section 31 acquires the action information from the reception part 22.

The second processing portion 42 detects the predefined action through the acquisition of the action information (Step SP301: YES), and identifies the guest who took the predefined action on the basis of the location information concerning the sensor 11 at that time. The second processing portion 42 calculates an action point by analyzing, on the basis of the location information and the action information, an action of the guest to determine interest degrees of the guest with respect to the living room 5A, allotting them to each of the interest degree items (i.e., “Life Proposal”, Interior”, and “Furniture and Equipment”), and representing them in a numerical form.

For example, the predefined action of sitting on a sofa is defined in advance to be worth three action points for “Life Proposal”, one action point for “Interior”, and two action points for “Furniture and Equipment”. The predefined action of switching on or off a television is defined in advance to be worth two action points for “Life Proposal”, one action point for “Interior”, and three action points for “Furniture and Equipment”. The second processing portion 42 adds three action points to “Life Proposal”, one action point to “Interior”, and two action points to “Furniture and Equipment” concerning the living room 5A in the interest degree information (the user data 56) concerning the guest who took the predefined action of sitting on a sofa.

When adding an action point, the second processing portion 42 may add a weight to each interest degree item on the basis of the basic point calculated for the same observable area. For example, when adding an action point, the second processing portion 42 may add an additional point to an interest degree item allotted with basic points equal to or greater than a threshold (e.g., four points on a five-point scale) concerning the living room 5A.

The second processing portion 42 continues the processing of adding an action point until the exit of the guest from the living room 5A is detected on the basis of location information from the location sensor SAL1 (Step SP303: YES).

In the example shown in FIG. 8, the action points are: eight points for “Life Proposal”;

four points for “Interior”; and four points for “Furniture and Equipment” concerning the living room 5A, respectively.

### Calculation Process of Individual Point

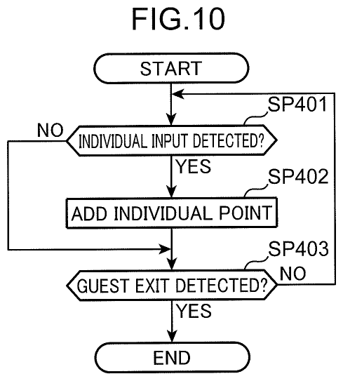

FIG. 10 is a flowchart showing a calculation process of an individual point that constitutes a part of the interest degree information concerning the guest.

In a case where a conversation with the guest during the guidance of the guest to the living room 5A or the like gives the host an impression that the guest is particularly interested in or curious about a certain interest degree item, the host may operate the operation part 66 of the tablet 12 to input an individual point to that interest degree item concerning the living room 5A.

The data processing part 61 of the tablet 12 sends individual information, which is input information input from the operation part 66, from the sending part 63 to the server device 10.

The reception part 22 of the server device 10 receives the individual information from the tablet 12. The acquisition section 31 acquires the individual information from the reception part 22.

The third processing portion 43 detects the input of the individual point through the acquisition of the individual information (Step SP401: YES), and calculates an individual point by allotting the interest degrees of the guest with respect to the living room 5A to each of the interest degree items (i.e., “Life Proposal”, Interior”, and “Furniture and Equipment”), and representing them in the numerical form on the basis of the individual information. Specifically, the third processing portion 43 adds the individual point included in the individual information to the interest degree item included in the individual information.

The third processing portion 43 continues the processing of adding an individual point until the exit of the guest from the living room 5A is detected on the basis of location information from the location sensor SAL1 (Step SP403: YES).

In the example shown in FIG. 8, the individual points concerning the living room 5A are: one point for “Life Proposal”; zero points for “Interior”; and zero points for “Furniture and Equipment”, respectively.

Subsequent to the living room 5A, the host guides the guest to the kitchen 6A and the bedroom 7A in turn, as well. The interest degree analysis section 33 executes the calculation processes of the basic point, the action point, and the individual point concerning the kitchen 6A and the bedroom 7A in the same manner as the living room 5A.

### Rest Proposal Process

The rest determination section 34 of the server device 10 determines whether an action of the guest in the housing exhibition site satisfies a predetermined rest condition or not, and sets rest information for the guest when the action satisfies the rest condition.

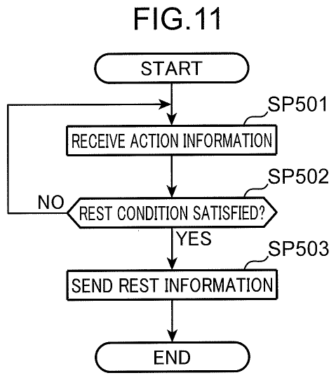

FIG. 11 is a flowchart showing a rest setting process executed by a rest determination section 34.

While the guest observes the inside of the housing exhibition site, the sensor 11 periodically sends action information indicative of an action item (a walking period, a walking distance, an average walking speed, and the number of steps, and the like) of the guest to the server device 10.

The reception part 22 of the server device 10 receives the action information sent from the sensor 11. The acquisition section 31 acquires the action information from the reception part 22.

When receiving the action information (Step SP501: YES), the rest determination section 34 determines whether the action item indicated in the action information satisfies the predetermined rest condition in Step SP502. For example, a reference value is set for each of the walking period, the walking distance, the average walking speed, and the number of steps. The rest determination section 34 determines that the rest condition is satisfied when two or more action items of the guest exceed the respective reference values. In the case that guests form a group, the rest determination section 34 determines that the rest condition is satisfied when action items concerning at least one guest included in the group exceed the respective reference values.

When determining that the rest condition is satisfied (Step SP502: YES), in Step SP503, the rest determination section 34 sends rest information for proposing a rest to the tablet 12 carried by the host in charge of the guidance of the guest. Specifically, the output section 35 outputs the rest information, and the sending part 23 sends the rest information input from the output section 35 to the tablet 12. The data processing part 61 of the tablet 12 displays the rest information received by the reception part 62 on the display part 65. The host proposes that the guest have a rest in accordance with the rest information displayed on the display part 65 of the tablet 12.

### Output Process of Determination Result

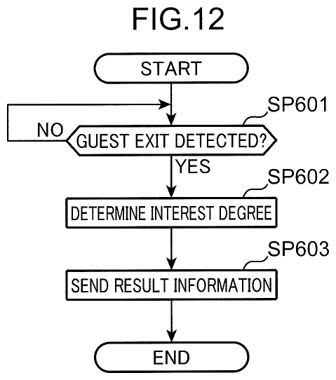

FIG. 12 is a flowchart showing an output process of result information indicative of a result of an interest degree determination concerning the guest.

When the guest exits the model house 3A under the guidance of the host, the data processing part 21 of the server device 10 detects the exit of the guest from the model house 3A on the basis of location information concerning the sensor 11 received from the location sensor SAE in the entrance 4A.

When detecting the exit of the guest from the model house 3A (Step SP601: YES), the interest degree analysis section 33 determines an interest degree of the guest in the model house 3A on the basis of the user data 56 concerning the guest in Step SP602. Specifically, the interest degree analysis section 33 estimates an interest degree of the guest in accordance with a total point which is obtained by summing basic points, action points, and individual points at each of observable areas and at each of interest degree items with respect to the model house 3A.

In the example shown in FIG. 8, the total point concerning “Life Proposal” is 34, the total point concerning “Interior” is 16, and the total point concerning “Furniture and Equipment” is 17, respectively. Therefore, the interest degree analysis section 33 estimates that the guest is most interested in “Life Proposal” of the model house 3A.

In the example shown in FIG. 8, the total point concerning the living room 5A is 25, the total point concerning the kitchen 6A is 22, and the total point concerning the bedroom 7A is 18, respectively. Therefore, the interest degree analysis section 33 estimates that the guest is most interested in the living room 5A of the model house 3A.

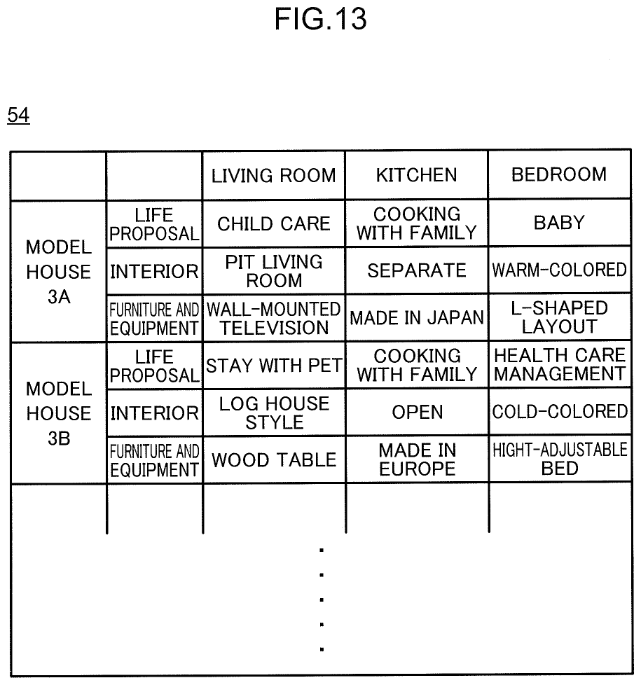

FIG. 13 is a table showing exemplary related data 54 stored in the storage part 24. The related data 54 has a plurality of keywords classified into the observable areas and the interest degree items for each of the model houses 3 provided in the housing exhibition site. In the example shown in FIG. 13, only one keyword is set for each item. However, a plurality of keywords may be set for a single item. The interest degree analysis section 33 determines that two model houses 3 which have a greater number of the same keywords have a higher concept similarity. In the example shown in FIG. 8, the interest degree analysis section 33 determines that the guest is most interest in the living room 5A of the model house 3. In this case, the interest degree analysis section 33 searches for another living room 5 having the largest number of the same keywords as the living room 5A, and proposes a model house 3 including the living room 5 as a subsequent candidate model house 3 to which the guest is guided.

Next, in Step SP603, the interest degree analysis section 33 sends result information indicative of a result of the interest degree determination in Step SP602 to the tablet 12 carried by the host in charge of the guidance of the guest. Specifically, the output section 35 outputs the result information, and the sending part 23 sends the result information input from the output section 35 to the tablet 12. The data processing part 61 of the tablet 12 displays the result information received by the reception part 62 on the display part 65. The host sees the result information displayed on the display part 65 of the tablet 12 and determines the subsequent model house 3 to which the guest is guided. If the subsequent model house 3 set in advance by the order setting section 32 and that set by the result information are different from each other, a subsequent model house 3 is determined at the discretion of the host.

### SUMMARY

In the interest degree determination system 1 of the embodiment, the sensor (first sensor) 11 detects physiological information concerning the guest, the location sensors (second sensors) SAL1, SAK1, SAB1 detect a location of the guest in the model house 3A, and the action sensors (third sensors) SAL2, SAL3, SAK2, SAK3, SAB2, SAB3 detect a predefined action of the guest in the model house 3A. Subsequently, the interest degree analysis section (analysis part) 33 included in the server device 10 determines, on the basis of the physiological information, the location information, and the action information received from these sensors, an interest degree of the guest with respect to the model house 3A. Accordingly, interest degree information (user data 56) indicative of an interest degree of the guest with respect to the model house 3A can be accurately collected by the server device 10.

Further, in the interest degree determination system 1 of the embodiment, the sending part (first sending part) 23 of the server device 10 sends the interest degree information to the tablet (operation terminal) 12 carried by the host, and the display part 65 of the tablet 12 displays the interest degree information received from the server device 10. Accordingly, a guidance service satisfying the guest can be stably provided by the host seeing the interest degree information concerning the guest displayed on the display part 65.

Further, in the interest degree determination system 1 of the embodiment, the first processing portion 41 included in the interest degree analysis section 33 calculates basic point (first point information) by allotting the interest degree of the guest with respect to the living room 5A to each of the interest degree items on the basis of the physiological information detected by the sensor 11 during a period in which the descriptive monitor (first presentation device) MAL presents description concerning the living room 5A to the guest, and representing them in a numerical form. Accordingly, interest degree information indicative of an interest degree of the guest with respect to the model house A at each of the interest degree items can be accurately generated by the interest degree analysis section 33.

Further, in the interest degree determination system 1 of the embodiment, the second processing portion 42 included in the interest degree analysis section 33 calculates action point (second point information) by analyzing, on the basis of the location information and the action information, an action of the guest in each of the observable areas to determine interest degrees of the guest with respect to model house 3A, and allotting them to each of the observable areas and each of the interest degree items, and representing them in the numerical form. Accordingly, interest degree information indicative of an interest degree of the guest with respect to the model house 3A at each of the observable areas and interest degree items can be accurately generated by the interest degree analysis section 33.

Further, in the interest degree determination system 1 of the embodiment, the second processing portion 42 adds a weight to each of the interest degree items on the basis of the basic points when adding the action points. Accordingly, an interest degree of the guest can be noticeably reflected to interest degree information which is generated by the interest degree analysis section 33.

Further, in the interest degree determination system 1 of the embodiment, individual information (input information) that is input by the host is sent from the tablet 12 to the server device 10, and a third processing portion 43 included in the interest degree analysis section 33 calculates individual point (third point information) by allotting the interest degree of the guest with respect to the model house 3A to each of the interest degree items and representing them in the numerical form on the basis of the individual information received from the tablet 12. Accordingly, interest degree information indicative of an interest degree of the guest with respect to the model house 3A at each of the interest degree items can be accurately generated by the interest degree analysis section 33.

Further, in the interest degree determination system 1 of the embodiment, the interest degree analysis section 33 adds, to result information (the interest degree information), information indicative of an interest degree item in which the guest is estimated to be most interested among the interest degree items. This enables the host who guides the guest to identify the interest degree item in which the guest is estimated to be most interested from the result information displayed on the display part 65 of the tablet 12. Accordingly, a satisfying guidance service can be provided to the guest.

Further, in the interest degree determination system 1 of the embodiment, the interest degree analysis section 33 adds, to the result information, information indicative of another model house 3 having a concept similar to a certain model house 3A to which the guest was guided. This enables the host to identify another model house 3 conceptually similar to the certain model house 3 in which the guest is interested from the result information displayed on the display part 65 of the tablet 12. Accordingly, a satisfying guidance service can be provided to the guest by guiding the guest to the another model house 3.

Further, in the interest degree determination system 1 of the embodiment, the order setting section (guidance order setting part) 32 sets a guidance order of the model houses 3 for the guest on the basis of the physiological information detected by the sensor 11 during a period in which the descriptive monitor (second presentation device) MW presents a description concerning a plurality of model houses 3 to the guest. Accordingly, further satisfying guidance service can be provided to the guest by setting a guidance order of the model houses 3 in a descending order of interest degree of the guest therein based on the physiological information.

Further, in the interest degree determination system 1 of the embodiment, the rest determination section 34 sets rest information for the guest if the action of the guest satisfies a predetermined rest condition. Accordingly, the server device 10 sends the rest information to the tablet 12, and the tablet 12 displays the rest information on the display part 65, which enables the host to propose a rest for the guest. Consequently, further satisfying guidance service can be provided to the guest.

Features of the embodiment described above are summarized hereinafter.

An interest degree determination system according to an aspect of the present invention is an interest degree determination system for determining an interest degree of the guest with respect to the building in a service allowing a guest to observe an inside of at least one building, and includes a server device, a first sensor that detects physiological information concerning the guest and sends the physiological information to the server device, a second sensor that detects a location of the guest in the building and sends location information indicative of the location to the server device, and a third sensor that detects a predefined action of the guest in the building and sends action information indicative of the action to the server device. The server device includes a first reception part that receives the physiological information, the location information, and the action information, and an analysis part that determines, on the basis of the physiological information, the location information, and the action information received by the first reception part, an interest degree of the guest with respect to the building, and outputs interest degree information indicative of the interest degree.

In this configuration, the first sensor detects the physiological information concerning the guest, the second sensor detects a location of the guest in the building, and the third sensor detects a predefined action of the guest in the building. The analysis part included in the server device determines, on the basis of the physiological information, the location information, and the action information received from the sensors, an interest degree of the guest with respect to the building. Accordingly, interest degree information indicative of an interest degree of the guest with respect to the building can be accurately collected by the server device.

In the above configuration, an operation terminal permitted to be operated by a host may be further provided, and the server device may further include a first sending part that sends the interest degree information to the operation terminal, and the operation terminal may include a second reception part that receives the interest degree information, and a display part that displays the interest degree information received by the second reception part.

In this configuration, the first sending part of the server device sends the interest degree information to an operation terminal carried by the host, and the display part of the operation terminal displays the interest degree information received from the server device. Accordingly, a satisfying guidance service can be stably provided to the guest by the host seeing the interest degree information concerning the guest displayed on the display part.

In the above configuration, a first presentation device that presents to the guest descriptions which concern the building and are respectively classified into a plurality of interest degree items may be further provided, and the analysis part may have a first processing section that generates first point information by allotting the interest degree of the guest with respect to the building to each of the interest degree items on the basis of the physiological information detected by the first sensor during a period in which the first presentation device presents descriptions to the guest, and representing them in a numerical form.

In this configuration, the first processing section included in the analysis part generates first point information by allotting the interest degree of the guest with respect to the building to each of the interest degree items on the basis of the physiological information detected by the first sensor during the period in which the first presentation device presents the descriptions concerning the building to the guest, and representing them in the numerical form. Accordingly, interest degree information indicative of an interest degree of the guest with respect to the building at each of the interest degree items can be accurately generated by the analysis part.

In the above configuration, the building includes a plurality of observable areas, and the analysis part may further have a second processing section that generates second point information by analyzing, on the basis of the location information and the action information, an action of the guest in each of the observable areas to determine interest degrees of the guest with respect to the building, and allotting them to each of the observable areas and each of the interest degree items, and representing them in the numerical form.

In this configuration, the second processing section included in the analysis part generates the second point information by analyzing, on the basis of the location information and the action information, an action of the guest in each of the observable areas to determine interest degrees of the guest with respect to the building, and allotting them to each of the observable areas and each of the interest degree items, and representing them in the numerical form. Accordingly, interest degree information indicative of an interest degree of the guest with respect to the building at each of the observable areas and interest degree items can be accurately generated by the analysis part.

In the above configuration, the second processing section may add a weight to each of the interest degree items on the basis of the first point information in the generation of the second point information.

In this configuration, the second processing section adds a weight to each of the interest degree items on the basis of the first point information in the generation of the second point information. Accordingly, an interest degree of the guest can be noticeably reflected to the interest degree information generated by the analysis part.

In the above configuration, the operation terminal may further include an operation part for allowing the host to input input information, the input information being information indicative of an interest degree of the guest with respect to the building and allotted to each of the interest degree items, and a second sending part that sends the input information input by a way of the operation part to the server device, the first reception part may further receive the input information, the analysis part may further have a third processing section that generates third point information by allotting the interest degree of the guest with respect to the building to each of the interest degree items and representing them in the numerical form on the basis of the input information received by the first reception part.

In this configuration, the input information input by the host is sent from the operation terminal to the server device, and the third processing section included in the analysis part generates third point information by allotting the interest degree of the guest with respect to the building to each of the interest degree items and representing them in the numerical form on the basis of the input information received by the operation terminal. Accordingly, interest degree information indicative of an interest degree of the guest with respect to the building at each of the interest degree items can be accurately generated by the analysis part.

In the above configuration, the analysis part may allot the interest degree of the guest with respect to the building to each of the interest degree items, and adds, to the interest degree information, information indicative of an interest degree item in which the guest is estimated to be most interested among the interest degree items.

In this configuration, the analysis part adds, to the interest degree information, information indicative of the interest degree item in which the guest is estimated to be most interested among the interest degree items. This enables the host who guides the guest to identify the interest degree item in which the guest is estimated to be most interested with reference to the interest degree information displayed on the display part of the operation terminal. Accordingly, a satisfying guidance service can be provided to the guest.

In the above configuration, the at least one building may include a plurality of buildings having concepts similar to or different from each other, the analysis part may add, to the interest degree information to be sent to the operation terminal when the host guides the guest to a certain building, information indicative of another building having a concept similar to the certain building.

In this configuration, the analysis part adds, to the interest degree information, the information indicative of another building having a concept similar to the certain building to which the guest was guided. This enables the host to identify another building having a concept similar to the certain building in which the guest is interested with reference to the interest degree information displayed on the display part of the operation terminal. Accordingly, a satisfying guidance service can be provided to the guest by guiding the guest to the another building.

In the above configuration, the at least one building may include a plurality of buildings, and the interest degree determination system may further include a second presentation device that presents to the guest a description concerning the buildings, wherein the server device further includes a guidance order setting part that sets, on the basis of the physiological information detected by the first sensor during a period in which the second presentation device presents the description to the guest, a guidance order of the buildings for the guest.

In this configuration, the guidance order setting part sets a guidance order of the buildings to the guest on the basis of the physiological information detected by the first sensor during the period in which the second presentation device presents the description of the buildings for the guest. Accordingly, further satisfying guidance service can be provided to the guest by setting the guidance order of the buildings in a descending order of the interest degree of the guest therein based on the physiological information.

In the above configuration, the server device may further include a rest determination part that determines whether an action of the guest satisfies a rest condition or not, and sets rest information for the guest if the action satisfies the rest condition.

In this configuration, the rest determination part sets rest information for the guest if the action of the guest satisfies a rest condition. Accordingly, the server device sends the rest information to the operation terminal, and the operation terminal displays the rest information on the display part, which enables the host to propose a rest for the guest. Consequently, further satisfying guidance service can be provided to the guest.

The server device according to an aspect of the present invention is a server device included in an interest degree determination system for determining an interest degree of the guest with respect to the building in a service that allows a guest to observe an inside of at least one building. The server device includes a reception part that receives, from a first sensor for detecting physiological information concerning the guest and sending the physiological information, the physiological information, receives, from a second sensor for detecting a location of the guest in the building and sending location information indicative of the location, the location information, and receives, from a third sensor for detecting a predefined action of the guest in the building and sending action information indicative of the action, the action information, and an analysis part that determines, on the basis of the physiological information, the location information, and the action information received by the reception part, an interest degree of the guest with respect to the building, and outputs interest degree information indicative of the interest degree.

In this configuration, the analysis part included in the server device determines an interest degree of the guest with respect to a building on the basis of the physiological information received from the first sensor, the location information received from the second sensor, and the action information received from the third sensor. Accordingly, interest degree information indicative of an interest degree of the guest with respect to the building can be accurately collected by the server device.

A program according to an aspect of the present invention is a program causing a computer mounted in a server device included in an interest degree determination system for determining, in a service allowing a guest to observe an inside of at least one building, an interest degree of the guest with respect to the building to function as: acquisition means that acquires, physiological information from a first sensor which detects the physiological information of the guest and sends the physiological information, location information from a second sensor which detects a location of the guest in the building and sends the location information indicative of the location, and action information from a third sensor which detects a predefined action of the guest in the building and sends the action information indicative of the action; and analysis means that determines, on the basis of the physiological information, the location information, and the action information acquired by the acquisition means, an interest degree of the guest with respect to the building, and outputs interest degree information indicative of the interest degree.

In this configuration, the analysis means determines the interest degree of the guest with respect to the building on the basis of the physiological information received from the first sensor, the location information received from the second sensor, and the action information received from the third sensor. Accordingly, interest degree information indicative of an interest degree of the guest with respect to the building can be accurately collected by the server device.

An interest degree determination method according to an aspect of the present invention is an interest degree determination method for determining, in a service allowing a guest to observe an inside of at least one building, an interest degree of the guest with respect to the building, and includes: detecting physiological information of the guest by a first sensor to send the physiological information to a server device; detecting a location of the guest in the building by a second sensor to send location information indicative of the location to the server device; detecting a predefined action of the guest in the building by a third sensor to send action information indicative of the action to the server device; and allowing the server device to: receive the physiological information, the location information, and the action information; and determine, on the basis of the received physiological information, location information, and action information, an interest degree of the guest with respect to the building and generate interest degree information indicative of the interest degree.

In this configuration, the server device determines the interest degree of the guest with respect to the building on the basis of the physiological information received from the first sensor, the location information received from the second sensor, and the action information received from the third sensor. Accordingly, interest degree information indicative of an interest degree of the guest with respect to the building can be accurately collected by the server device.

1. An interest degree determination system for determining, in a service allowing a guest to observe an inside of at least one building, an interest degree of the guest with respect to the building, the interest degree determination system comprising:
a server device;
a first sensor that detects physiological information concerning the guest and sends the physiological information to the server device;
a second sensor that detects a location of the guest in the building and sends location information indicative of the location to the server device; and
a third sensor that detects a predefined action of the guest in the building and sends action information indicative of the action to the server device, wherein
the server device includes:
a first reception part that receives the physiological information, the location information, and the action information; and
an analysis part that determines, on the basis of the physiological information, the location information, and the action information received by the first reception part, an interest degree of the guest with respect to the building, and outputs interest degree information indicative of the interest degree.

2. The interest degree determination system according to claim 1,
further comprising:
an operation terminal permitted to be operated by a host, wherein
the server device further includes:
a first sending part that sends the interest degree information to the operation terminal, and
the operation terminal includes:
a second reception part that receives the interest degree information; and
a display part that displays the interest degree information received by the second reception part.

3. The interest degree determination system according to claim 2,
further comprising:
a first presentation device that presents to the guest descriptions which concern the building and are respectively classified into a plurality of interest degree items, wherein the analysis part has:
a first processing section that generates first point information by allotting the interest degree of the guest with respect to the building to each of the interest degree items on the basis of the physiological information detected by the first sensor during a period in which the first presentation device presents the descriptions to the guest, and representing them in a numerical form.

4. The interest degree determination system according to claim 3, wherein
the building includes a plurality of observable areas, and
the analysis part further has:
a second processing section that generates second point information by analyzing, on the basis of the location information and the action information, an action of the guest in each of the observable areas to determine interest degrees of the guest with respect to the building, allotting them to each of the observable areas and each of the interest degree items, and representing them in the numerical form.

5. The interest degree determination system according to claim 4, wherein
the second processing section adds a weight to each of the interest degree items on the basis of the first point information in the generation of the second point information.

6. The interest degree determination system according to any one of claims 3 to 5, wherein the operation terminal further includes:
an operation part for allowing the host to input input information, the input information being information indicative of an interest degree of the guest with respect to the building and allotted to each of the interest degree items; and
a second sending part that sends the input information input by a way of the operation part to the server device,
the first reception part further receives the input information,
the analysis part further has:
a third processing section that generates third point information by allotting the interest degree of the guest with respect to the building to each of the interest degree items and representing them in the numerical form on the basis of the input information received by the first reception part.

7. The interest degree determination system according to any one of claims 1 to 6, wherein the analysis part
allots the interest degree of the guest with respect to the building to each of the interest degree items, and
adds, to the interest degree information, information indicative of an interest degree item in which the guest is estimated to be most interested among the interest degree items.

8. The interest degree determination system according to any one of claims 2 to 7, wherein
the at least one building includes a plurality of buildings having concepts similar to or different from each other,
the analysis part adds, to the interest degree information to be sent to the operation terminal when the host guides the guest to a certain building, information indicative of another building having a concept similar to the certain building.

9. The interest degree determination system according to any one of claims 2 to 8, wherein the at least one building includes a plurality of buildings,
further comprising:
a second presentation device that presents to the guest a description concerning the buildings, wherein
the server device further includes:
a guidance order setting part that sets, on the basis of the physiological information detected by the first sensor during a period in which the second presentation device presents the description to the guest, a guidance order of the buildings for the guest.

10. The interest degree determination system according to any one of claims 1 to 9, wherein
the server device further includes:
a rest determination part that determines whether an action of the guest satisfies a rest condition or not, and sets rest information for the guest if the action satisfies the rest condition.

11. A server device included in an interest degree determination system for determining, in a service that allows a guest to observe an inside of at least one building, an interest degree of the guest with respect to the building, the server device comprising:
a reception part that
receives, from a first sensor for detecting physiological information concerning the guest and sending the physiological information, the physiological information,
receives, from a second sensor for detecting a location of the guest in the building and sending location information indicative of the location, the location information, and
receives, from a third sensor for detecting a predefined action of the guest in the building and sending action information indicative of the action, the action information; and
an analysis part that determines, on the basis of the physiological information, the location information, and the action information received by the reception part, an interest degree of the guest with respect to the building, and outputs interest degree information indicative of the interest degree.

12. A program causing a computer mounted in a server device included in an interest degree determination system for determining, in a service allowing a guest to observe an inside of at least one building, an interest degree of the guest with respect to the building to function as:
acquisition means that acquires,
physiological information from a first sensor which detects the physiological information of the guest and sends the physiological information,
location information from a second sensor which detects a location of the guest in the building and sends the location information indicative of the location, and
action information from a third sensor which detects a predefined action of the guest in the building and sends the action information indicative of the action; and
analysis means that determines, on the basis of the physiological information, the location information, and the action information acquired by the acquisition means, an interest degree of the guest with respect to the building, and outputs interest degree information indicative of the interest degree.

13. An interest degree determination method for determining, in a service allowing a guest to observe an inside of at least one building, an interest degree of the guest with respect to the building, comprising:
detecting physiological information of the guest by a first sensor to send the physiological information to a server device;
detecting a location of the guest in the building by a second sensor to send location information indicative of the location to the server device;
detecting a predefined action of the guest in the building by a third sensor to send action information indicative of the action to the server device; and
allowing the server device to:
receive the physiological information, the location information, and the action information; and
determine, on the basis of the received physiological information, location information, and action information, an interest degree of the guest with respect to the building and generate interest degree information indicative of the interest degree.
